(19) United States
(12) Patent Application Publication
(10) Pub. No.: US 2010/0291629 A1
(43) Pub. Date: Nov. 18, 2010

(54) USE OF DELEUCOCYTATION FILTERS FOR DEFENSIN PURIFICATION

(75) Inventors: Chantal Fournier-Wirth, Clapiers (FR); Joliette Coste, Montpellier (FR)

Correspondence Address: CANTOR COLBURN LLP, 20 Church Street, 22nd Floor, Hartford, CT, 06103, US

(73) Assignee: ETABLISSEMENT FRANCAIS DU SANG, Paris (FR)

(21) Appl. No.: 12/294,500

(22) PCT Filed: Mar. 27, 2007
(86) PCT No.: PCT/FR2007/051023

§ 371 Date: Aug. 5, 2010

(30) Foreign Application Priority Data
Mar. 28, 2006 (FR) 0651067

(51) Int. Cl. C12P 21/00; C07K 14/47

(52) U.S. Cl. 435/71.1; 530/344

(57) ABSTRACT

The invention concerns the use of deleucocytation filters for defensin purification. More precisely, the invention concerns a method for purifying defensins, including the following steps: passing a blood sample on a deleucocytation filter; detaching the cells retained on the deleucocytation filter, inducing exocytosis of the defensins by those cells.

### TECHNICAL FIELD

The invention concerns the purification of α-defensins, peptides synthesised by human neutrophils.

This purification is performed using deleukocytation (or leukodepletion) filters which retain the leukocytes when preparing various labile blood products.

Thanks to a well-suited extraction and purification protocol, it is possible to obtain large quantities of active peptides with many applications due to their bactericidal, virucidal, cytotoxic, immunomodulator and chemotactic properties.

This source of α-defensins is particularly well suited given the simplicity and good yield of the protocol applied. Moreover, it is an economical source, insofar as deleukocytation filters are waste products destined to be destroyed.

### PRIOR ART

Mammals are constantly exposed to a wide range of micro-organisms. They are rarely infected, however, thanks to the protective barrier role played by the skin and epithelia, and the presence of antimicrobial substances (1). If they get across the barrier, these invasive pathogens are controlled and eliminated later by the host's immune system. Mammals have developed two types of immunity: “innate”, which is non-clonal and non-specific, and “adaptive”, which is inducible and specific to antigens.

Innate (constitutive) immunity is the host's first line of defence, which is quickly mobilized when a microbial invasion is detected. It is based on the recruitment and/or activation of leukocytes (granulocytes, monocytes/macrophages, etc.) capable of fighting off pathogens, and on the release and/or activation of a variety of extracellular humoral mediators (complement components, cytokines, antimicrobial substances, etc).

Adaptive immunity is induced when lymphocytes are activated in response to the antigen presented by antigen-presenting cells (APCs), notably dendritic cells (DCs). The receptors present on the T-lymphocytes recognise the antigenic epitopes linked to the major hisocompatibility complex (MHC) present on the surface of the APCs. CD8+ T-lymphocytes, activated after recognising the antigenic epitopes linked to class I MHCs (on most cells with nuclei in the organism), differentiate into cytotoxic lymphocytes, which directly kill cells infected by pathogens. CD4+ T-lymphocytes, activated after recognising antigenic epitopes linked to the class II MHC (on macrophages, dendritic cells, B-lymphocytes, etc.), develop into T helper cells which, by producing various cytokines, trigger the activation of B-lymphocytes (maturation into plasma cells and stimulation of antibody secretion) and help the phagocytes to eliminate the pathogens.

Innate and adaptive immune responses thus work in concert to eliminate microbial invaders.

Antimicrobial substances include microbicidal chemical molecules (hydrogen peroxide, nitric oxides, etc.) and a wide range of antimicrobial proteins (defensins, histatins, cathelicidins, etc.) with many types of biological activity that help the host's defences.

Among them, defensins are cationic peptides with 29 to 42 amino acids containing 6 invariant cystein residues which form 3 intramolecular disulphide bridges (2, 3). These disulphide bridges are essential to antimicrobial and cytotoxic activities.

The two main families of defensins in vertebrates, α- and β-defensins, differ in the length of the peptide segments between the 6 cystein residues and in the pairing of these residues into disulphide bridges.

Currently, 6 human α-defensins (29 to 35 amino acids) have been identified. α-defensins 1-4 are also called HNP 1-4 (Human Neutrophil Peptides) because they were originally isolated in neutrophils (granulocytes) (4). HNP 1-3 only differ in the nature of their first amino acid (5). The other two, HD-5 and -6, are mainly expressed by intestinal Paneth cells (6).

These defensins have all been purified, sequenced, tested for their antimicrobial activities and analysed by NMR and crystallography. Their cDNA has also been cloned and sequenced (6, 7).

The three α-defensins, HNP 1-3, account for 99% of the defensin content of neutrophils and 30 to 50% of azurophil granule proteins (8). The quantity of defensins measured is 3 to 5 μg/million neutrophils (9). The concentration of HNP 1-3 in the plasma of healthy subjects is approximately 40 ng/ml, but can reach 1 to 100 μg/ml in cases of severe infections (7).

All the defensins characterised so far have direct antimicrobial effects (10). The antimicrobial spectrum is very broad and includes Gram (+) bacteria (Staphylococcus aureus, etc.) and Gram (−) bacteria (Escherichia coli, etc.), mycobacteria, many fungi and certain enveloped viruses (HIV, VSV, HSV, Influenza A, WNV, etc.) (1-4, 6, 10).

The mechanism of action is not yet totally clear (11). The interaction between defensins and micro-organisms appears to destabilise and disturb the cell membranes and therefore to increase cell permeability and small molecule fixation. The polar topology of defensins, with hydrophobic regions separated from charged regions, enables them to be inserted into microbial cell membranes which contain more negatively charged residues than the mammal cell membranes. This interaction appears to be at the origin of the formation of multiple pores in the targeted cell membrane (1, 2).

It was very recently demonstrated that α-defensins can inhibit the lethal toxin of Bacillus anthracis under physiological conditions and protect against the fatal consequences of anthrax.

Moreover, α-defensins have chemotactic effects.

Phagocytes, including neutrophils and monocytes/macrophages, are the first effector cells in the innate antimicrobial defence after pathogens enter the host (1). These phagocytes have to be recruited at the sites of invasion to fight against the pathogen. Leukocyte recruitment occurs in the presence of chemokines and other chemotactic factors.

α-defensins have a chemotactic effect on monocytes, naive T-lymphocytes and immature dendritic cells (DCs), although no specific receptor has been identified (1-3). DCs take part in innate immunity and can act directly by phagocyting and killing the pathogen or produce many mediators after activation (cytokines, chemokines, etc.).

α-defensins are also inductors of inflammatory mediators, inducing the degranulation of mast cells and the release of histamine. The products of mast cell granules increase the influx of neutrophils, and defensins could therefore indirectly promote the recruitment of phagocytic neutrophils at the site of inflammation. Degranulation, here of the recruited neutrophils, causes the release of defensins and therefore a positive regulatory loop (1).

Moreover, HNP 1-3 may increase the expression of pro-inflammatory cytokines TNF-α and IL1 in monocytes and decrease that of IL10 which, at the site of microbial infection, could also amplify the inflammatory response (1).

Once recruited at the site of the infection, the phagocytes become activated and work with many effector molecules to destroy the microbial invader. Human α-defensins can also directly activate phagocytes to facilitate phagocytosis. Lastly, they participate in regulating activation of the conventional channel of the complement system (1).

In cases of systemic infections, the plasma concentrations of α-defensins can reach 100 μg/ml, which can interfere with the production of glucocorticoids (powerful immunosuppressants). Defensins may therefore also facilitate antimicrobial immunity by inhibiting the production of these immunosuppressant mediators.

In the area of innate immunity, the in vivo contribution of defensins thus consists in:
deactivating or killing micro-organisms directly;
facilitating phagocytosis;
promoting the recruitment of phagocytes, mast cells and DCs;
increasing the production of pro-inflammatory cytokines;
suppressing anti-inflammatory and immunosuppressant mediators;
regulating activation of the complement.

Furthermore, defensins are adaptive immunity activators.

Implementation of antimicrobial adaptive immunity at the sites of invasion starts when the microbial antigens are taken on by immature DCs (1). While processing the antigens, the immature DCs evolve in response to the endogenous mediators and microbial products to become mature DCs able to migrate to lymphoid tissue and stimulate naive Ag-specific T-cells. Defensins, released in large quantities in response to microbial and inflammatory stimuli, contribute to the recruitment of immature DCs at the sites of invasion and to their maturation. Thus, they play a critical role in the induction of suitable immune responses.

Moreover, α- and β-defensins are chemotactic for lymphocytes and therefore participate in the recruitment of CD4+ and CD8+ effector T-cells at the sites of microbial infection. HNP 1-3 are also present in the nucleus of peripheral blood T-cells, which suggests that they may deeply regulate T-lymphocyte functions.

Lastly, the immunostimulant activity of α- and β-defensins has been demonstrated in vivo (1). How can defensins promote adaptive immunity in the context of an infection? Defensins facilitate the handling and processing of Ags by dendritic cells. They encourage antigenic presentation by inducing the maturation of DCs directly or indirectly (through the production of TNF-α and IL 1 by monocytes/macrophages). Lastly, defensins facilitate the recruitment of CD4+ and CD8+ memory cells in infected tissues and therefore contribute to the effector phase of antimicrobial immune responses.

Given the multiple properties of defensins, their purification has been the subject of many studies.

These started in 1985 with the extraction of HNP 1-3 from human neutrophils (4). The protocol consisted in isolating granulocytes from leukapheresis, then extracting a sediment rich in granules which then underwent chromatography (Biogel P-10 column/HPLC) to purify the three α-defensins. Antimicrobial activity (against S. aureus, P. aeruginosa and E. coli), antifungal activity (against C. neofermentants) and antiviral activity (against HSV) of the purification product were reported. Electronic microscope work has also been used to locate HNP 1-3 in azurophil granules. Simultaneously, the primary structure of these defensins, comprising 29 (HNP 2) or 30 (HNP 1 and 3) amino acids and differing by the nature of the first amino acid only, has been described (5).

In 1994, the preparation and extraction of granules from a leukapheresis bag, neutrophils obtained from “buffy coat” (leukocyte-platelet layer) or whole blood were reported. The defensins were then purified by chromatography using the granules (12).

According to Shiomi et al., the concentration of HNP 1-3 in human plasma is equal to 400 ng/ml vs. 13 μg/ml in whole blood (12). The quantity of HNP 1-3 for 1.10⁶ neutrophils is estimated to be between 3 and 5 μg (8, 9).

Defensins have the particularity of being able to be secreted after neutrophil stimulation by PMA (phorbol myristate acetate). It has thus been demonstrated that, at high concentrations of PMA (1 μg/ml), 8% of the defensins are released (9). The defensin release profile, depending on the PMA dose, is correlated (although weaker) to that of β-glucuronidase and elastase, other azurophil granule markers. One possible explanation for the poor detection of defensins in the extracellular environment after PMA stimulation could be the fact that defensins have a strong affinity for cell surfaces. In the secretion process, these peptides may therefore be adsorbed or incorporated into the neutrophil cell membrane.

Neutrophil treatment with IL8 (Interleukine 8) at 50 ng/ml in vitro leads to their degranulation and defensin release at 10% of the defensin content in 2.10⁶ neutrophil granules. As a comparison, and according to the analyses using Western Blot images (scan+NIH image), treatment with fMLP (10⁻⁷ M) releases 17% of the content, while using a PMA (2 ng/ml)/ionomycin (500 nM) mixture 23% (13).

The secretory properties of neutrophils were more closely studied by Borregaard's team in 2002 (Faurschou et al., 14). The release of α-defensins and the traditional markers of various types of granules was studied in response to different stimulations of isolated neutrophils (30.10⁶): Ionomycin (1 μM), Cyt B (Cytochalasin B) (5 μM)/fMLP (formylmethionyl-leucyl-phenylalanin) (1 μM), PMA (5 μg/ml) or fMLP (10 nM). Under all conditions, α-defensins and myeloperoxidase (azurophil granules) exocytosis was observed at the same level, while that of lactoferrine was slightly higher and that of gelatinase was the highest. For α-defensins, the best results have been obtained with stimulation by ionomycin or by combining CytB/fMLP. In both cases, an exocytosis rate of approximately 20% was observed.

Document WO 2005/049637 describes a method for purifying defensins from white blood cells based on a relatively complicated protocol: release of defensins in an acid solution, extraction from said solution, then purification steps, notably using chromatography.

There is currently an obvious need for new defensins purification protocols. These molecules are of major therapeutic interest, notably as broad-spectrum antibiotics, for which the chemical synthesis of active forms is reputed to be particularly delicate.

The technical problem that the present invention proposes to solve is therefore to provide a source and a protocol for extracting large quantities of defensins easily, with good yield and at a moderate cost.

### DETAILED DESCRIPTION OF THE INVENTION

Thus, and according to a first aspect, the invention concerns the use of deleukocytation filters for defensin purification.

Deleukocytation filters are used to filter blood while preparing labile blood products (LBP) such as PRBC (packed red blood cells), PC (platelet concentrate), FFP (fresh frozen plasma) or PCM (platelet concentrate mixture). Their role is to remove the leukocytes from these blood products, where neutrophils make up the majority population, and which are trapped by said filters.

Deleukocytation filters are available in the market. These may be, for example, “filters for the deleukocytation of standard platelet concentrate mixtures using Buffy Coat”, sold by the Pall Corp. These are polyester multi-layer filters providing leukocyte retention by affinity, the exact mechanism of action (by ligand or by charge effect) being kept secret by the manufacturers.

In the protocols for preparing blood products, deleukocyation filters are to be destroyed. They are thus sources of large quantities of neutrophils, and therefore defensins, which are both enriched and highly economical. The present invention is based on the concept of biological recycling. Defensins could be a new molecule resulting from blood product fractionation.

Furthermore, these deleukocytation filters are part of a tightly controlled chain for obtaining “blood-derived drugs”. They benefit from experience in transfusion, notably good practices for preparation, product traceability demands and haemovigilance controls. These deleukocytation filters are thus a sanitarily safe source of defensins.

Document WO 93/16201 proposed using cellular retentate from a blood sample trapped on a deleukocytation filter to analyse viral contamination. For this, the cells on the filter are lysed to release the viral genome or viral antigens and the appropriate tests (PCR or antibodies) are carried out on this material.

Furthermore, document WO 02/14560 called for the use of deleukocytation filters to analyse the genomic DNA of the cellular retentate. According to this protocol, the cells are lysed in situ. The DNA remains attached to the filter while washing out the cell debris and is then eluted in purified form.

However, no document describes the use of these filters for the preparation of significant quantities of active peptide materials.

Preferentially, the defensins are of human origin, i.e. the deleukocytation filters are those retrieved after treating human blood.

As mentioned above, the neutrophil cells thus retrieved only contain α-defensins, more precisely α-defensins 1 to 4 (HNP 1-4), 99% of which are α-defensins 1 to 3 (HNP 1-3). Moreover, due to the similarity of the sequences and structures of these 3 defensins, conventional purification protocols, for example those based on molecular weight or antigenicity, produce copurification of these three peptides, HNP 1-3 .

The term “purification”, used in the context of the present invention, has a meaning similar to the terms “extraction” or “isolation”. It entails enriching the biological material in active defensins, whose concentration is thus increased.

Starting with the raw material consisting of deleukocytation filters rich in neutrophils, one or more successive purification steps can be applied.

According to a second aspect, the invention concerns a defensin purification procedure. In a first step, it is characterised in that a blood extract is passed through a deleukocytation filter and said filter is retrieved.

Advantageously, the blood extract is of human origin.

The blood extract may be constituted of raw blood. Preferably, it is a leukocyte-platelet layer, also called a “buffy coat”.

In a further step, the filter is washed at least once, preferably 5 to 6 times, using a saline buffer. Typically, the buffer is PBS (Phosphate Buffer Saline). Advantageously, this deleukocytation filter treatment is performed in a sterile environment, for example under a laminar air flow hood. The purpose of this step is to release the carrier neutrophils in their defensin granules.

The volume of washing buffer is gathered and is called “lysate”. This lysate contains the cells or cell fragments detached from the filter under the action of the saline buffer.

The following step consists in having defensins secreted by the cells.

Defensin secretion stimulation is known to persons skilled in the art. Advantageously, it is performed in the presence of Cyt B (Cytochalasin B) and fMLP (N-formylmethionyl-leucyl-phenylalanine), added to a micromolar concentration, preferentially between 1 and 10 μM.

This exocytosis can be carried out on the whole lysate, but is advantageously applied to the cell pellet obtained after centrifuging the lysate. This centrifugation is typically performed for a few minutes at approximately 2,500 rpm.

To avoid the breakdown of defensins which are no longer protected by the granules after secretion, protease inhibitors are advantageously added to the reaction mixture.

After halting the reaction and centrifugation (200 g for a few minutes), the defensins secreted are retrieved from the supernatant.

To enrich the fraction thus obtained in α-defensins 1 to 3 (HNP 1-3), the supernatant can be passed through an immunoprecipitation column containing a specific antibody for HNP 1-3. These peptides are first captured on this column due to the antigen-antibody affinity and are then eluted in purified form.

Of course, later purification steps such as chromatography (gel filtration, HPLC, etc.) may be carried out for the desired degree of purity.

The originality of this protocol therefore lies in direct chemical treatment of deleukocytation filters and avoids heavy purification steps on cells or organelles. Indeed, exocytosis is performed directly on cells from deleukocytation filters without granulocytes and azurophil granules being isolated. Thus, the extraction procedure applied is very simple, constituting a strong point in terms of automation.

Moreover, this procedure provides excellent yield: it appears that using a deleukocytation filter which, as been said, comes from the filtration of a pool of platelet concentrates corresponding to 5 or 6 “buffy coats”, makes it possible to obtain at least 0.5 μg, advantageously between 1 μg and 2 μg, and even up to 5 μg of purified HNP 1-3 defensins.

### Examples of Embodiments

The purpose of the examples of embodiments described below is to provide a further illustration of the present invention in one of its privileged embodiments. These examples of embodiments, however, are in no case exhaustive.

### LEGEND FOR THE FIGURES

FIG. 1: Deleukocytation filter setup.

FIG. 2: Western Blot analysis of purified HNP 1-3 defensins from deleukocytation filters.

FIG. 3: SELDI-TOF-MS analysis of purified HNP 1-3 defensins from deleukocytation filters.

FIG. 4: SELDI-TOF-MS analysis (zoom) of purified HNP 1-3 defensins from deleukocytation filters.

### 1/ PCM Preparation:

Platelet concentrate mixtures (PCMs) are traditionally prepared using the “buffy coat” method. A whole blood sample from a blood donor is taken following good transfusion practices and then stored for 15 to 20 hours at room temperature before preparing the different blood products. The whole blood is then centrifuged at 4,000 rpm for 20 minutes at 20° C. and separated into red corpuscles, plasma and the leukocyte-platelet layer called the “buffy coat” (containing platelets, leukocytes and 45 g of plasma). After separation, 5 or 6 compatible “buffy coats” are gathered and centrifuged at low speed (1,700 rpm for 11 minutes at 20° C.). The supernatant containing the platelets is then filtered to retrieve the leukocytes and transferred into a final storage bag which is then distributed as leukodepleted “PCM”.

All of these steps are carried out in a closed circuit using a disposable preparation kit containing several bags. The different mixed “buffy coats” are gathered through sterile connections between the different unit bags. The deleukocytation filter used in the final step before transfer into the storage bag constitutes the initial source of defensins. In practice, this is a deleukocytation filter sold by the Pall Corporation with part number Pall ATSBC2PSF “AutoStop BC Pall”.

### 2/ Deleukocytation Filter Treatment:

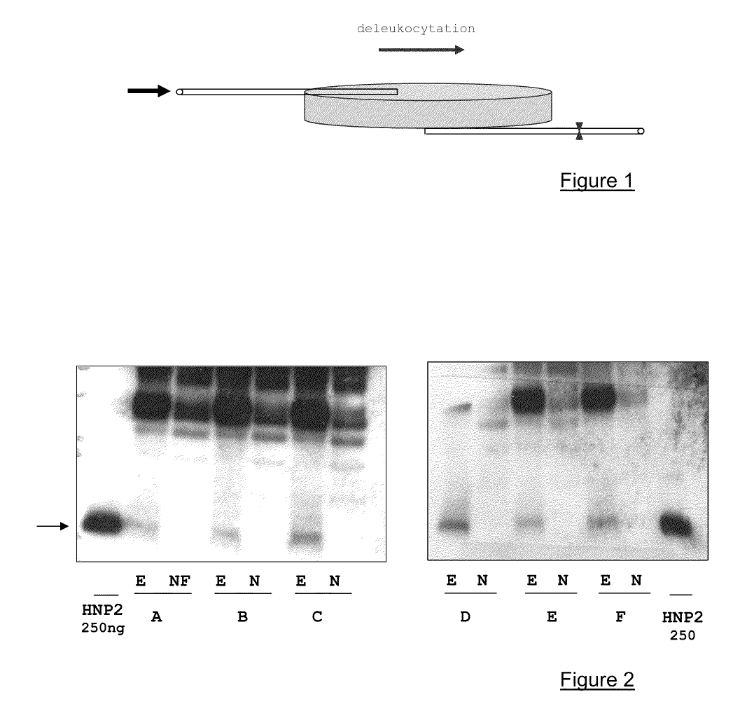

A deleukocytation filter, installed on a closed blood product treatment circuit, is illustrated in FIG. 1.

After performing the final PCM filtration, the filter is isolated from the kit by 2 sterile seals on the tubing upstream and downstream from it. The filter, made of rigid plastic, cannot be opened.

The filter is then treated directly under sterile conditions under a laminar air flow hood: solution A (Phosphate Buffer Saline (PBS 1×), 3 ml) is injected using a syringe inserted into the upstream tubing. The filter is rinsed by aspiration, running the syringe back and forth 6 times. The lysate is gathered in a tube.

### 3/ Defensin Purification:

Three short steps are performed successively:
centrifugation

The lysate (2 ml) obtained after washing the filter is centrifuged at 2,500 rpm for 10 minutes at 10° C.
exocytosis

The supernatant obtained is eliminated. Stimulation is performed on the cell pellet after adding protease inhibitors (Complete Tablets, Roche; 112 μl) and 160 μl of Krebs-Ringer buffer (130 mM NaCl, 5 mM KCl, 1.27 mM MgSO₄, 0.95 mM CaCl₂, 5 mM glucose, 10 mM NaH₂PO₄/Na₂HPO₄, pH 7.4), with treatment lasting 5 minutes at 37° C. with Cytochalasin B (Sigma, 2.10⁻⁵ M; 104 μl or 5.53 μM) followed by incubation for 15 minutes at 37° C. in the presence of N-formylmethionyl-leucyl-phenylalanine (fMLP, Sigma, 11.4.10⁻⁶ M; 43 μl or 1.17 μM). The reaction is halted by dilution with cold KRP buffer (531 μl). After centrifugation (200 g for 6 minutes), the supernatant obtained is divided into four 240-microlitre tubes and may be stored at −80° C.

This protocol is adapted from that described by Faurshou et al. (14).
Immunoprecipitation

Each aliquot obtained after exocytosis is treated by immunoprecipitation to purify α-defensins HNP 1-3.

Immunoprecipitations are performed using the μMacs Protein G Microbeads system (Miltenyi Biotec). Purified Mouse anti-human α-defensin 1-3 Monoclonal Antibody (BD Pharmingen) is used for capture.

In practice, 240 μl of supernatant (exocytosis product) are mixed with 5 μl of anti-HNP 1-3 antibody (0.2 mg/ml), 50 μl of magnetic beads and 700 μl of lysis buffer (150 mM NaCl, 1% Triton X100, 50 mM Tris HCl, pH 8). The whole is incubated in ice for 75 minutes. The mixture is then deposited on a μMACS column prehydrated with 200 μl of lysis buffer. The column is washed 4 times with 200 μl of “High Salt Buffer” washing buffer (500 mM NaCl, 1% NP-40, 50 mM Tris HCl, pH 8), then once with 100 μl of “Low Salt Buffer” washing buffer (20 mM Tris HCl, pH 7.5). Then, 20 μl of elution buffer (50 mM Tris HCl, pH 6.8, 1% SDS) are preheated to 95° C., applied and left in contact for 10 minutes. Elution is performed with 50 μl of elution buffer preheated to 95° C.

The eluate obtained with each immunoprecipitation column (45 microlitres in the end, on average) is stored at −20° C. before analysis. 30 μl are used for the Western Blot analysis in the presence of 10 μl of deposit buffer (NuPage Invitrogen LDS 4×).

### 4/ Analysis of the HNP 1-3 Eluates Obtained After Immunoprecipitation:

### 4.1. Reference

A commercial purified human HNP 2 defensin (>95% HPLC/3370.95 Da, Sigma) was used as the reference for the tests.

A sequence check (chemical NH2 terminal sequencing) was performed by the Protein Microsequencing and Analysis platform at the Institut Pasteur in Paris.

### 4.2. Western Blot Detection

The eluted proteins are separated under non-reductive conditions by mono-dimensional electrophoresis in acrylamide gel (NuPAGE Bis-tris 12% acrylamide, Invitrogen) and transferred onto a nitrocellulose membrane (Hybond ECL, Amersham). Purified Mouse anti-human α-defensin 1-3 Monoclonal Antibody (0.2 μg/μl, BD Pharmingen) is used for HNP1-3 defensin recognition. A chemoluminescent immunodetection kit (WesternBreeze, Invitrogen) is used according to the supplier's recommendations for visualisation.

The primary structure of α-defensins was described in 1985 by R. Lehrer's team (5). These proteins comprise 29 (HNP 2) or 30 (HNP 1 and 3) amino acids which only differ by the nature of the first amino acid. Thus, the commercial monoclonal antibody chosen is used for immunoprecipitation and detection of these three defensins, but cannot be used to discriminate amongst them.

Six deleukocytation filters (A to F) for the platelet concentrate mixtures (PCMs) prepared using the “buffy coat” method for purifying α-defensins were treated (FIG. 2). After immunoprecipitation with the anti-HNP 1-3 monoclonal antibody, each eluate obtained (E) was analysed with Western Blot. The non-fixed fractions (NF) are used as the negative control. Synthetic HNP 2 defensin (Sigma) is used as the positive control for the Western Blot.

Using the anti-HNP 1-3 monoclonal antibody, the presence of proteins at the expected masses is detected in the eluates obtained after immunoprecipitation (E). These proteins are not detected in the first fractions obtained when the lysate is run through the column (non-fixed fractions, NF).

A quantitative estimate of the signals was performed using image analysis software (SigmaGel Software) on all of the profiles obtained with Western Blot. Thus, it was estimated that rapid exocytosis treatment of the cells obtained after the centrifugation of 2 ml of lysate (from one filter wash) releases between 0.5 μg and 1.5 μg of purified HNP 1-3 defensins.

### 4.3. SELDI-TOF-MS Spectrometry Detection (Ciphergen Biosystem)

Ciphergen's SELDI-TOF-MS ProteinChip Technology (Palo Alto, Calif.) (www.ciphergen.com) combines protein capture on active chemical surfaces and time-of-flight mass spectrometry analysis (TOF-MS). It is used to analyse proteins directly from complex biological fluids and requires very small quantities for analysis (microsampling). This technology provides separation, detection and analysis of proteins on the femtomolar level. It improves discovery of proteins lower than 30 kDa and is insensitive to their isoelectric point.

The originality of the ProteinChip platform lies in the existence of different arrays for separating and capturing proteins in relation to their chemical or biochemical properties (hydrophobic, anionic, cationic, etc.) and in the data analysis software used for comparing protein profiles.

This technology has various applications: studying the differential expression of proteins, molecular recognition, protein purification and characterisation, marker validation and identification.

This technology was used by Zhang et al. (15) to study human HNP 1-3 defensins as anti-HIV antiviral molecules and, more recently, by Albrethsen et al. (16) in a proteomic approach aimed at studying HNP 1-3 expression in colon cancer.

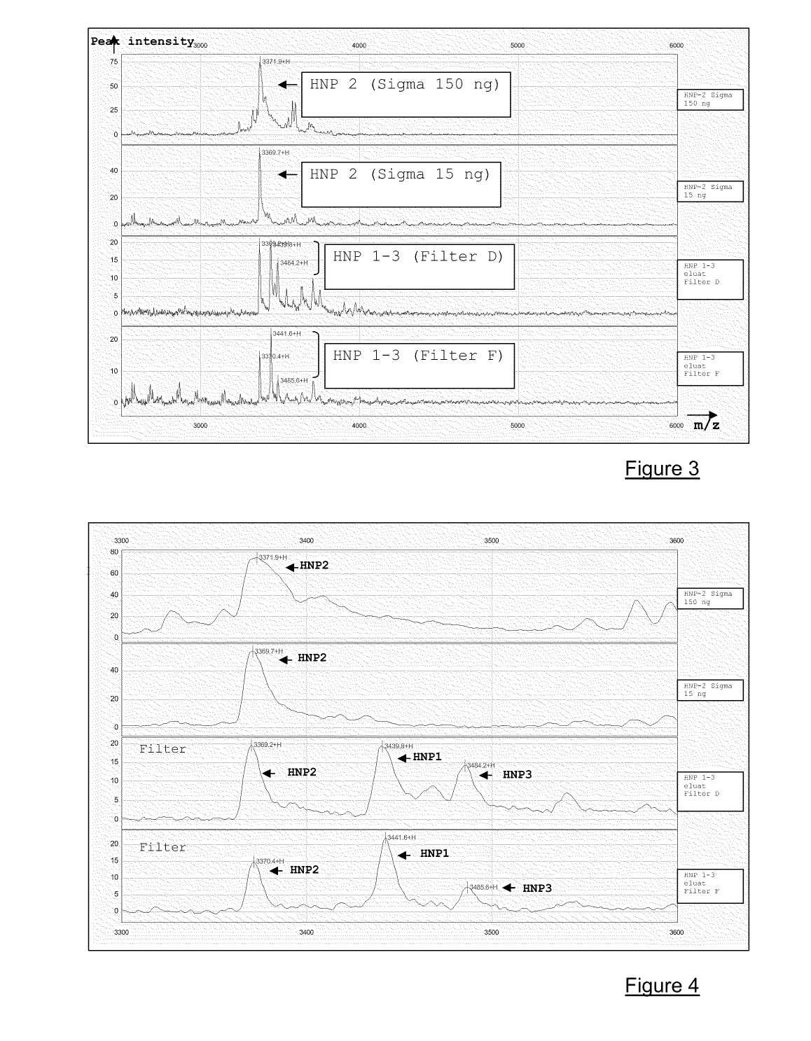

The immunoprecipitation eluate (3 μl) is analysed directly on an NP20 array (NP20 ProteinChip array, normal phase, Ciphergen). After drying (20 minutes), the non-fixed proteins are eliminated by two successive washings and a drying time of 5 to 10 minutes is applied before adding the matrix (SPA, 50% acetonitrile, 0.5% TFA) for protein desorption. The array is then placed in the analysis platform (Ciphergen ProteinChip Reader PBSII). The proteins complexed in the matrix are desorbed by the laser and their time-of-flight is proportional to their mass/charge ratio (m/z). The peaks detected are represented in FIG. 3 and zoomed in FIG. 4.

All of the peaks were analysed after calibration using the “All in One” peptide molecular weight standards calibration mix (Ciphergen Biosystems) under the conditions laid down by the supplier.

The following reading criteria were applied: laser intensity 180/detector sensitivity 9/High Mass 10,000 optimized from 1,000 to 7,500/Focus mass: 3,400.

It was thus confirmed that proteins were present with mass/charge ratios of 3370, 3440 and 3485 in the immunoprecipitation eluates. These peptides correspond to defensins HNP 2, HNP 1 and HNP 3, respectively.

These results are in agreement with the spectra obtained by SELDI-TOF-MS in the articles by Zhang et al. (3372/3442/3486; 15) and Albrethsen et al. (3372/3443/3486; 16).

The results obtained thus validate the possibility of purifying alpha HNP 1-3 defensins in platelet concentrate mixtures from deleukocytation filters.

### BIBLIOGRAPHY

1. Yang D., Biragyn A., Hoover D. M., Lubkowski J., Oppenheim J. J.; Multiple roles of antimicrobial defensins, cathelicidins, and eosinophil-derived neurotoxin in host defense. Annu Rev Immunol 2004; 22:181-215.
2. Ganz T.; Defensins: antimicrobial peptides of innate immunity. Nat Rev Immunol 2003; 3:710-720.
3. Yang D., Biragyn A., Kwak L. W., Oppenheim J. J.; Mammalian defensins in immunity: more than just microbicidal. Trends Immunol 2002; 23:291-296.
4. Ganz T., Selsted M. E., Szklarek D., Harwig S. S., Daher K., Bainton D. F., Lehrer R. I.; Defensins. Natural peptide antibiotics of human neutrophils. J Clin Invest 1985; 76:1427-1435.
5. Selsted M. E., Harwig S. S., Ganz T., Schilling J. W., Lehrer R. I.; Primary structures of three human neutrophil defensins. J Clin Invest 1985; 76:1436-1439.
6. Lehrer R. I., Lichtenstein A. K., Ganz T.; Defensins: antimicrobial and cytotoxic peptides of mammalian cells. Annu Rev Immunol 1993; 11:105-128.
7. Ganz T., Lehrer R. I.; Defensins. Pharmacol Ther 1995; 66:191-205.
8. Shiomi K., Nakazato M., Ihi T., Kangawa K., Matsuo H., Matsukura S.; Establishment of radioimmunoassay for human neutrophil peptides and their increases in plasma and neutrophil in infection. Biochem Biophys Res Commun 1993; 195:1336-1344.
9. Ganz T.; Extracellular release of antimicrobial defensins by human polymorphonuclear leukocytes. Infect Immun 1987; 55:568-571.
10. Ganz T., Lehrer R. I.; Antimicrobial peptides of vertebrates. Curr Opin Immunol 1998; 10:41-44.
11. Selsted M., Ouellette A. J.; Mammalian defensins in the antimicrobial immune response. Nature Immunol. 2005; 6:551-557.
12. Harwig S. S., Ganz T., Lehrer R. I.; Neutrophil defensins: purification, characterization, and antimicrobial testing. Methods Enzymol 1994; 236:160-172.
13. Chertov O., Michiel D. F., Xu L., Wang J. M., Tani K., Murphy W. J., Longo D. L., et al.; Identification of defensin-1, defensin-2, and CAP37/azurocidin as T-cell chemoattractant proteins released from interleukin-8-stimulated neutrophils. J Biol Chem 1996; 271:2935-2940.
14. Faurschou M., Sorensen O. E., Johnsen A. H., Askaa J., Borregaard N.; Defensin-rich granules of human neutrophils: characterization of secretory properties. Biochim Biophys Acta 2002; 1591:29-35.
15. Zhang L., Yu W., He T., et al.; Contribution of human alpha-defensin 1, 2, and 3 to the anti-HIV-1 activity of CD8 antiviral factor. Science. 2002; 298:995-1000.
16. Albrethsen J., Bogebo R., Gammeltoft S., Olsen J., Winther B., Raskov H.; Upregulated expression of human neutrophil peptides 1, 2 and 3 (HNP 1-3) in colon cancer serum and tumours: a biomarker study. BMC Cancer. 2005; 5:8.

1. Defensin purification method wherein the defensins are purified from a deleukocytation filter.

2. Defensin purification method as claimed in claim 1, including the following steps:
running a blood extract through a deleukocytation filter;
releasing the cells trapped on the deleukocytation filter;
inducing defensin exocytosis by these cells.

3. Defensin purification method as claimed in claim 2, wherein the blood extract is a leukocyte-platelet layer.

4. Defensin purification method as claimed in claim 2, wherein the release of the cells trapped on the deleukocytation filter is achieved by washing with a saline buffer.

5. Defensin purification method as claimed in claim 2, wherein exocytosis is induced on the cell pellet obtained after centrifugation of the washing product from the deleukocytation filter.

6. Defensin purification method as claimed in claim 2, wherein exocytosis is induced using Cyt B and fMLP.

7. Defensin purification method as claimed in claim 2, wherein, after exocytosis, centrifugation is performed and the supernatant is gathered.

8. Defensin purification method as claimed in claim 7 wherein, after exocytosis, immunoprecipitation is performed on the supernatant.

9. Defensin purification method as claimed in claim 2, wherein blood extract is of human origin and the purified defensins are HNP 1-3.
